(19) United States
(12) Patent Application Publication
(10) Pub. No.: US 2011/0038899 A1
(43) Pub. Date: Feb. 17, 2011

(54) Pharmaceutical Solutions and Method for Solublilizing Therapeutic Agents

(76) Inventors: Garry Thomas Gwozdz, Jim Thorpe, PA (US); Andrew Loxley, Philadelphia, PA (US); Mark Mitchnick, East Hampton, NY (US)

Correspondence Address: LICATA & TYRRELL P.C., 66 E. MAIN STREET, MARLTON, NJ, 08053, US

(21) Appl. No.: 12/933,701

(22) PCT Filed: Mar. 27, 2009
(86) PCT No.: PCT/US09/38518

§ 371 Date: Oct. 14, 2010

Related U.S. Application Data
(60) Provisional application No. 61/040,281, filed on Mar. 28, 2008.

(51) Int. Cl. A61K 9/00; A61K 31/5513; A61K 38/00; A61K 31/7052; A61K 31/573; A61K 31/565; A61K 31/436; A61K 31/337; A61K 38/12

(52) U.S. Cl. 424/400; 514/221; 514/1.1; 514/52; 514/180; 514/182; 514/291; 514/449; 514/21.1

(57) ABSTRACT

Pharmaceutical solutions containing hydrophobic or lipophilic therapeutic agents and methods for producing the same are provided. Pharmaceutical solutions of the invention are produced by dissolving the therapeutic agent in one or more tocopherols or tocotrienols and one or more alcohols or glycols. These solutions are used to produce pharmaceutical compositions.

This patent application claims the benefit of priority from U.S. Application Ser. No. 61/040,281, filed Mar. 28, 2008, teachings of which are herein incorporated by reference in their entirety.

### BACKGROUND OF THE INVENTION

A vast number of potential therapeutic agents are discovered each year, many of which are water insoluble or poorly water soluble. For such hydrophobic compounds, direct injection may be impossible or highly dangerous, and can result in hemolysis, phlebitis, hypersensitivity, organ failure and/or death. Such compounds are termed by pharmacists as “lipophilic”, “hydrophobic”, or in their most insoluble form, “amphiphobic”.

A few examples of therapeutic agents in these categories are ibuprofen, diazepam, griseofulvin, cyclosporin, cortisone, proleukin, etoposide and paclitaxel. (Kagkadis et al. PDA J. Pharm. Sci. Tech. 1996 50(5):317-323; Dardel Anaesth. Scand. 1976 20:221-24; Sweetana and Akers PDA J. Pharm. Sci. Tech. 1996 50(5):330-342).

Administration of chemotherapeutic agents is particularly problematic. Most of these agents are poorly soluble and thus are difficult to deliver in aqueous solvents and to supply at therapeutically effective levels. Further, water-soluble, chemotherapeutic agents are generally taken up by both cancer and non-cancer cells, making such agents non-specific and oftentimes unacceptably toxic.

For therapeutic agents that cannot be formulated as an aqueous solution, emulsions have oftentimes provided a cost-effective and therapeutically acceptable alternative. However, it is difficult to render emulsions sterile and/or endotoxin free for intravenous injection. Oils typically used for pharmaceutical emulsions include saponifiable oils from the family of triglycerides, for example, soybean oil, sesame seed oil, cottonseed oil, safflower oil and the like (Hansrani, et al. J. Parenter. Sci. Technol. 1983 37:145-150). One or more surfactants are used to stabilize the emulsion, and excipients are added to render the emulsion more biocompatible, stable and less toxic. Lecithin from egg yolks or soybeans is a commonly used surfactant. Sterile manufacturing can be accomplished by sterilization of all the components before manufacture, followed by aseptic technique in all stages of manufacture. Improved ease of manufacture and assurance of sterility is obtained by terminal sterilization following sanitary manufacture, either by heat or by filtration. However, terminal sterilization by heat or filtration treatments is not suitable for all emulsions.

Vitamin E emulsions have been disclosed. For example, injectable vitamin E emulsions are described by Hidiroglou and Karpinski (Brit. J. Nutrit. 1988 59:509-518) for dietary supplementation in sheep and for research on the pharmacokinetics of vitamin E and its derivatives. An injectable form of vitamin E for mice was prepared by Kato et al. (Chem. Pharm. Bull. 1993 41(3):599-604). Micellar solutions were formulated with TWEEN 80, BRIJ 58 and HCO-60. Isopropanol was used as a co-solvent, and was then removed by vacuum evaporation; the residual oil glass was then taken up in water with vortexing as a micellar suspension. An emulsion was also prepared by dissolving vitamin E with soy phosphatidycholine (lecithin) and soybean oil. Water was added and the emulsion prepared with sonication. Ethanol-free emulsions of alpha-tocopherol, stabilized by biocompatible surfactants, as a vehicle or carrier for therapeutic drugs is also disclosed in U.S. Pat. Nos. 6,667,048 and 6,660,286.

E-Ferol, a vitamin E emulsion for vitamin E supplementation and therapy in neonates was also disclosed by Alade et al. (Pediatrics (1986) 77(4):593-597). The surfactant mixture used to emulsify the 25 mg/mL vitamin E in E-Ferol was composed of 9% TWEEN 80 and 1% TWEEN 20. However, this supplement was not safe.

An alternative means of solubilizing low solubility compounds is direct solubilization in a non-aqueous milieu, for example, alcohols (such as ethanol), dimethylsulfoxide, and/or triacetin. For example, WO 95/11039 describes the use of vitamin E (100 mg), lecithin (20 mg), ethanol (100 mg) and EUTANOL (500 mg) as an injectable formulation of the immunosuppressant molecule cyclosporine (50 mg). U.S. Pat. No. 5,689,846 discloses various alcohol solutions of paclitaxel. U.S. Pat. No. 5,573,781 discloses the dissolution of paclitaxel in ethanol, butanol and hexanol and an increase in the antitumor activity of paclitaxel when delivered in butanol and hexanol as compared to ethanol.

WO 95/31217 discloses that tocopherols can be used as solvents and/or emulsifiers of drugs that are substantially insoluble in water, in particular for the preparation of topical formulations. The use of vitamin E-TPGS as an emulsifier in formulations containing high levels of α-tocopherol is mentioned and formulations for topical administration composed of a lipid layer (α-tocopherol), the drug and vitamin E-TPGS as an emulsifier in quantities of less than 25% w/w of the formulation.

WO 97/03651 discloses lipid vehicle drug delivery compositions that contain at least five ingredients: a therapeutic drug, vitamin E, an oil in which the drug and vitamin E are dissolved, a stabilizer (either phospholipid, a lecithin, or a poloxamer which is a polyoxyethylene-polyoxypropylene copolymer) and water.

Similarly, U.S. Pat. No. 6,962,691 teaches topical compositions composed of at least ten ingredients: alendronate sodium, povidone, povidone vinyl acetate, vitamin E, menthol, dimethyl isosorbide, acetone, ethanol, tetrafluoroethane and, dichlorodifluoromethane.

U.S. Pat. No. 4,393,073 also suggests vitamin E as an active ingredient in pharmaceutical compositions containing ethanol.

### SUMMARY OF THE INVENTION

An aspect of the present invention relates to a pharmaceutical solution comprising a therapeutic agent dissolved in one or more natural or synthetic tocopherols or tocotrienols, or any combination thereof and one or more alcohols or glycols, or any combinations thereof. In some embodiments, the tocopherol(s) and/or tocotrienol(s) is in an amount from about 30% to about 99% (w/w) and the alcohol(s) and/or glycol(s) is in an amount from about 1% to about 70% (w/w).

Another aspect of the present invention relates to methods for producing these pharmaceutical solutions.

Another aspect of the present invention relates to methods of treatment of a patient with these pharmaceutical solutions.

### DETAILED DESCRIPTION OF THE INVENTION

The present invention is directed to the use of one or more tocopherols and/or tocotrienols and one or more alcohols and/or glycols as pharmaceutically acceptable solvents for solubilizing therapeutic agents, in particular hydrophobic or lipophilic therapeutic agents. Advantageously, the resulting pharmaceutical solution is not an emulsion or vesicle, and can be used directly in the production of pharmaceutical compositions. Moreover, the combination of a tocopherol and/or a tocotrienol and an alcohol and/or glycol is much less irritating to the skin and/or mucous membranes than pure alcohol solutions and generally provides higher loading of a therapeutic agent than emulsions, liposomes, encapsulations, or cyclodextrins.

A solution in the context of the present invention is a homogeneous mixture composed of three or more substances. In such a mixture, a solute is dissolved in another substance, known as a solvent. In accordance with the present invention, a pharmaceutical solution is formed by dissolving a therapeutic agent in a tocopherol and/or a tocotrienol and an alcohol and/or glycol as solvents. In one embodiment of the present invention, the therapeutic agent is dissolved completely in the tocopherol and/or a tocotrienol and the alcohol and/or glycol solvents. In another embodiment of the present invention, the therapeutic agent may not be completely solubilized and thus is partially dissolved in the tocopherol and/or a tocotrienol and the alcohol and/or glycol solvents. In this embodiment, particulates of therapeutic agent may be present in the pharmaceutical solution. The resulting pharmaceutical solutions of either embodiment can be used in a variety of pharmaceutical compositions with various modes of administration.

The combination of tocopherol and/or a tocotrienol and alcohol and/or glycol is also useful in solubilizing at least in part amphiphobic therapeutic agents. In this embodiment, the solution acts as a transport phase through partial solubilization to increase the bioavailability of the amphiphobic therapeutic agent from a finely divided suspension of the agent.

Tocopherols and/or tocotrienols for use in accordance with the present invention include a family of natural and synthetic compounds, also known by the generic names tocols or vitamin E. Alpha-tocopherol is the most abundant and active form of this family of compounds, and it has the following chemical structure:

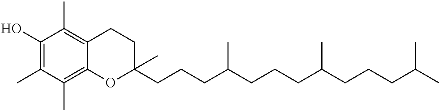

Other members of this family include beta-, gamma-, and delta-tocopherol, alpha-, beta-, gamma-, and delta-tocotrienols, tocopsoralen, alpha-tocopherol derivatives and/or analogs such as tocopherol acetate, phosphate, succinate, nicotinate and linoleate, as well as isomers thereof and esters thereof. Use of the phrase tocopherol(s) and/or tocotrienol(s) herein is meant to be inclusive of any member of this family alone or in combination. In one embodiment of the present invention the tocopherol(s) and/or tocotrienol(s) employed is alpha-tocopherol.

Examples of alcohol(s) for use in the present invention include, but are not limited to ethanol, propyl alcohol, butyl alcohol, pentanol, benzyl alcohol, and any isomers thereof, and any combinations thereof. Examples of glycols for use in the present invention include, but are not limited to ethylene glycol, propylene glycol, butylene glycol, pentylene glycol, and any isomers thereof, and any combinations thereof. In one embodiment of the present invention the alcohol is ethanol (ethyl alcohol). Preferred is use of an ethanol that is biocompatible in the sense that it is not toxic and does not cause any physiological or pharmacological effects. In this regard, the ethanol is desirably 180 to 200 proof ethanol, i.e., in the range of 90-100% ethanol. Advantageously, diluting a tocopherol or tocotrienol with an alcohol or glycol dramatically reduces the inherent viscosity of the tocopherol or tocotrienol thereby allowing for generation of sprayable formulations.

In accordance with the present invention, solutions of one or more tocopherols and/or tocotrienols and one or more alcohols and/or glycols are used in the solubilization of hydrophobic or lipophilic therapeutic agents thereby providing increased bioavailability of the therapeutic agent. In some embodiments, the tocopherol(s) and/or tocotrienol(s) is in an amount from about 30% to about 99% (w/w) and the alcohol(s) and/or glycol(s) is in an amount about 1% to about 70% (w/w).

As a non-limiting example, the solubility of Diazepam at room temperature is less than or equal to 6.67% in 190 proof ethanol. However, combining tocopherol and ethanol has been found to provide solubility of the Diazepam approaching the 10% level. By way of illustration, at 70% tocopherol: 30% ethanol (200 proof), Diazepam is soluble to greater than or equal to 8% and at 95% tocopherol: 5% ethanol (200 proof), Diazepam is soluble at greater than or equal to 9%.

Accordingly, preferred for some embodiments is that alpha-tocopherol and ethanol constitute 60% to 99% and 1% to 40%, respectively, of the pharmaceutical solution. In other embodiments, the alpha-tocopherol and ethanol constitute approximately 70% to 90% and 10% to 30%, respectively, of the pharmaceutical solution. In still other embodiments, the tocopherol and ethanol are used at ratios of approximately 95:5, 90:10, 85:15, 80-20, 75:25, 70:30, 65:35, or 60:40, respectively.

Pharmaceutical solutions of the present invention can be produced by dissolving any difficult to solubilize therapeutic agent (i.e., hydrophobic or lipophilic therapeutic agents) in one or more tocopherols and/or tocotrienols and one or more alcohols and/or glycols as pharmaceutically acceptable solvents. By therapeutic agents it is meant to be inclusive of, but is not limited to, small organic molecules, therapeutic peptides, non-peptides and nucleotides. Hydrophobic derivatives of water-soluble molecules such as lipid conjugates/prodrugs are also within the scope of therapeutic agents.

Exemplary hydrophobic or lipophilic therapeutic agents which can be solubilized in accordance with the present invention include, but are in no way limited to, steroids such as Dexamethasone, 17-beta-Estradiol; benzodiazepenes such as Diazepam, alpraxolam, bromazepam, chlordiazepoxidem, clonazepam, estazolam, flunitrazepam, flurazepam, lorazepam, lormetazepam, mexazolam, nitrazepam, oxazepam, temazepam, and triazolam; Rapamycin and analogues; Taxol (paclitaxel) and analogues; Actinomycin D; Prostaglandins (PGE1); Vitamin A; Probucol; Batimastat; Statins (HMG-CoA Reductase Inhibitors; Trapidil (and other anti-proliferative Growth Factor Inhibitors); Cytochalasin B; and microtubule binding agents such as epothilones, elutherobin and discodermolide. Pharmaceutical solutions and compositions formulated from the solutions may comprise one or more therapeutic agents in solution. Further, pharmaceutical compositions formulated from the pharmaceutical solutions of the present invention may further comprise one or more additional therapeutic agents in encapsulated or micronized (not dissolved) forms.

The present invention also provides for use of a combination of tocopherol and/or a tocotrienol and alcohol and/or glycol to solubilize at least in part amphiphobic therapeutic agents. In this embodiment, the solution acts as a transport phase through partial solubilization to increase the bioavailability of the amphiphobic therapeutic agent from a finely divided suspension of the agent.

Pharmaceutical solutions of the invention can be formulated into pharmaceutical compositions for administration to animals, preferably humans, via intravascular, oral, intramuscular, cutaneous and subcutaneous routes. Specifically, pharmaceutical compositions of the present invention can be administered by any of the following nonlimiting exemplary routes, intraabdominal, intraarterial, intraarticular, intracapsular, intracervical, intracranial, intraductal, intradural, intralesional, intralocular, intralumbar, intramural, intranasal, intraocular, intraoperative, intraparietal, intraperitoneal, intrapleural, intrapulmonary, intraspinal, intrathoracic, intratracheal, intratympanic, intrauterine, and intraventricular. The pharmaceutical compositions of the present invention can be nebulized using mechanical nebulizers or suitable aerosol propellants which are known in the art for pulmonary delivery of lipophilic compounds. The most suitable route in any given case will depend on the nature and severity of the condition being treated and on the nature of the particular therapeutic agent which is being used.

Pharmaceutical solutions of the instant invention are particularly useful in formulations to be administered to mucosal membranes, i.e. the nasal mucosa or lungs of a subject by any suitable means. For many therapeutic agents, administration via the nasal route provides for faster attainment of therapeutic levels of the therapeutic agent systemically. However, many therapeutic agents are so slightly soluble in water that a therapeutically effective amount cannot be dissolved in a volume of aqueous solvent that is amenable to application to a mucosal membrane. Use of a pharmaceutical solution of the present invention, however, provides for improved ability to dissolve hydrophilic and lipophilic therapeutic agents, thus providing a useful delivery system for administration of such agents to one or more mucosal membranes, including the nasal mucosal membranes. Such solutions can be administered via, for example, a metered dose inhaler or nebulizer, or in a mist sprayer.

The instant pharmaceutical solutions comprising a therapeutic agent, one or more tocopherols and/or tocotrienols and one or more alcohols and/or glycols can also be formulated into a pharmaceutical composition for injection by combining the instant pharmaceutical solution with, e.g., saline solution or water and a Vitamin E solubilizing agent such as Cremophor. Such pharmaceutical compositions may further contain other pharmaceutically acceptable additives such as, but not limited to, acidifying, alkalizing, buffering, chelating, complexing and solubilizing agents, antioxidants and antimicrobial preservatives, penetration enhancers, humectants, suspending and/or viscosity modifying agents, tonicity and wetting or other biocompatible materials.

For oral therapeutic administration, the instant pharmaceutical solutions can be combined with one or more carriers and used in the form of ingestible tablets, buccal tablets, troches, capsules, elixirs, suspensions, syrups, wafers, chewing gums, foods and the like. Such compositions and preparations should contain at least 0.1% of active compound. Tablets, troches, pills, capsules, and the like can also contain one or more of the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring. The above listing is merely representative and one skilled in the art could envision other binders, excipients, sweetening agents and the like. When the unit dosage form is a capsule, it can contain, in addition to materials of the above type, a liquid carrier, such as a vegetable oil or a polyethylene glycol. Various other materials can be present as coatings or to otherwise modify the physical form of the solid unit dosage form. For instance, tablets, pills, or capsules can be coated with gelatin, wax, shellac or sugar and the like.

A syrup or elixir can contain the instant pharmaceutical solution, sucrose or fructose as a sweetening agent, methyl and propylparabens as preservatives, a dye and flavoring such as cherry or orange flavor. Of course, any material used in preparing any unit dosage form should be substantially non-toxic in the amounts employed.

In addition, the instant pharmaceutical solution can be formulated into sustained-release preparations and devices including, but not limited to, those relying on osmotic pressures to obtain a desired release profile.

Formulations suitable for parenteral administration can be aqueous or non-aqueous injection solutions, which are generally isotonic with the blood of the intended recipient. These preparations can contain anti-oxidants, buffers, bacteriostats and solutes which render the formulation isotonic with the blood of the intended recipient. Aqueous and non-aqueous sterile suspensions can include suspending agents and thickening agents. The formulations can be presented in unit\dose or multi-dose containers, for example sealed ampoules and vials.

Formulations suitable for topical application to the skin can take the form of an ointment, cream, lotion, paste, gel, spray, aerosol, oil or other pharmaceutical formulation which accomplishes direct contact between the therapeutic agent and the skin. Topical formulations can also be prepared which are suitable for occlusive therapy.

Formulations in the forms of ointments, creams, lotions and pastes can generally have carriers in the forms of oleaginous bases (e.g., White Petrolatum and White Ointment); absorption bases formed by adding a water-in-oil emulsifying agent to an oleaginous base (e.g., Hydrophilic Petrolatum, AQUABASE, and AQUAPHOR); water-in-oil emulsion bases, prepared by adding water to an absorption base (e.g., HYDROCREAM, EUCERIN, NIVEA, and Cold Cream); oil-in-water emulsion bases (e.g., DERMABASE, UNIBASE, VELVACHOL, and hydrophilic ointment); and water soluble bases (e.g., polyethylene glycol ointment such as PEG 400-600 G or PEG 3350-400 G). Suitable carriers to produce a spray, gel, or aerosol are well-known in the art.

A carrier for topical application can also contain additional ingredients such as other carriers, moisturizers, penetration enhancers, humectants, emollients, dispersants, radiation blocking compounds, cleansing agents, anti-infective agents (e.g., antibiotics, fungicides, scabicides, or pediculicides), anti-inflammatory agents (e.g., corticosteroids), keratolytics (agents that soften, loosen, and facilitate exfoliation of the squamous cells of the epidermis), as well as other suitable materials that do not have a significant adverse effect on the activity of the topical composition. Additional ingredients can include, for example a sodium acid phosphate moisturizer, witch hazel extract, glycerine humectant, apricot kernal oil emollient, or corn oil dispersant. Other materials which can optionally be included in a topical composition include inositol or B-complex vitamins.

Formulations suitable for transdermal administration can be presented as discrete patches adapted to remain in intimate contact with the epidermis of the recipient for a prolonged period of time. Formulations suitable for transdermal administration can also be delivered by iontophoresis (see, for example, Pharmaceutical Research 3 (6):318 (1986)) and typically take the form of an optionally buffered aqueous solution. Formulations of the present invention are also suitable for delivery via microneedle delivery technology for cutaneous administration.

What is claimed is:

1. A pharmaceutical solution comprising one or more therapeutic agents dissolved in one or more tocopherols or tocotrienols and one or more alcohols or glycols.

2. The pharmaceutical solution of claim 1 wherein the one or more tocopherol or tocotrienol is 30% to 99% and the one or more alcohol or glycol is 1% to 70% of the volume of the pharmaceutical solution.

3. The pharmaceutical solution of claim 1 wherein the one or more tocopherols or tocotrienols is alpha-tocopherol.

4. The pharmaceutical solution of claim 1 wherein the one or more alcohols or glycols is ethanol.

5. A pharmaceutical solution consisting of a therapeutic agent dissolved in alpha-tocopherol and ethanol.

6. A pharmaceutical composition comprising the pharmaceutical solution of any of claims 1 through 5.

7. The pharmaceutical composition of claim 6 further comprising one or more additional therapeutic agents in encapsulated or micronized forms.

8. The pharmaceutical solution of any of claims 1 through 5 wherein the one or more therapeutic agents is partially dissolved in one or more tocopherols or tocotrienols and one or more alcohols or glycols.

9. A method for solubilizing a therapeutic agent comprising dissolving a therapeutic agent in one or more tocopherols or tocotrienols and one or more alcohols or glycols to form a pharmaceutical solution.

10. The method of claim 9, wherein the one or more tocopherol or tocotrienol is 30% to 99% and the one or more alcohol or glycol is 1% to 70% of the volume of the pharmaceutical solution.

11. The method of claim 9 wherein the one or more tocopherols or tocotrienols is alpha-tocopherol.

12. The method of claim 9 wherein the one or more alcohols or glycols is ethanol.

13. The method of any of claims 9 through 12 wherein the therapeutic agent is partially dissolved in one or more tocopherols or tocotrienols and one or more alcohols or glycols.
